(19) United States
(12) Patent Application Publication
(10) Pub. No.: US 2004/0092719 A1
(43) Pub. Date: May 13, 2004

(54) Isolation of immunoglobulin molecules that lack inter-heavy chain disulfide bonds

(76) Inventors: Eszter Birck-Wilson, Ashland, MA (US); Maria Day, Milford, MA (US)

Correspondence Address: GTC BIOTHERAPEUTICS, INC., 175 CROSSING BOULEVARD, SUITE 410, FRAMINGHAM, MA, 01702, US

(21) Appl. No.: 10/659,856

(22) Filed: Sep. 11, 2003

Related U.S. Application Data
(60) Provisional application No. 60/411,419, filed on Sep. 17, 2002.

(51) Int. Cl. C07K 16/18

(52) U.S. Cl. 530/387.1

(57) ABSTRACT

The current invention features methods for reliably and controllably separating immunoglobulin half antibodies from immunoglobulin whole antibodies, as well as purified immunoglobulin half antibody preparations and purified immunoglobulin whole antibody preparations while preserving biological activity. These dissociated half antibodies can be chromatographically separated from whole antibodies. There are four known subclasses of IgG molecules: IgG₁; IgG₂; IgG₃; and IgG₄. IgG₄ molecules differ from the other IgG isotypes in that the disulfide bonds that link the two heavy chain subunits together do not always form. Due to the non-covalent interactions that hold the heavy chain subunits together, the heterogeneity of IgG₄ molecules is not apparent following gel filtration of purified IgG₄ protein. However, when purified IgG₄ protein is separated by denaturing polyacrylamide gel electrophoresis (SDS-PAGE) under non-reducing conditions, two distinct protein species can be identified—whole antibody and “half-antibodies.”

### FIELD OF THE INVENTION

The current invention provides methodology allowing for the controlled separation of immunoglobulin half antibodies from “immunoglobulin whole antibodies” while preserving biological activity. More specifically the invention features methods for separating immunoglobulin half antibodies from immunoglobulin whole antibodies, as well as purified immunoglobulin half antibody preparations and purified immunoglobulin whole antibody preparations.

### BACKGROUND OF THE INVENTION

Immunoglobulin molecules such as IgA, IgD, IgE, IgG, and IgM molecules are multimeric proteins that participate in the vertebrate immune response. The basic structure of immunoglobulin molecules is tetrameric and consists of two light chain subunits and two heavy chain subunits; the heavy chain subunits are class specific and impart unique characteristics upon the different classes of immunoglobulin molecules. The four-chain structure of immunoglobulin molecules is held together by strong non-covalent interactions between the amino terminal half of each heavy chain subunit with a light chain subunit and between the carboxy terminal half of the two heavy chain subunits. Disulfide bonds further strengthen these interactions by creating links between both the heavy and light chain subunits and the two heavy chain subunits. It should also be noted for the purposes of the current invention that IgM has 10 heavy and 10 light chains, while IgA is mostly dimer, containing 4 chains of both the light and the heavy variety.

There are four known subclasses of IgG molecules: IgG₁; IgG₂; IgG₃; and IgG₄. IgG₄ molecules differ from the other IgG isotypes in that the disulfide bonds that link the two heavy chain subunits together do not always form. Due to the non-covalent interactions that hold the heavy chain subunits together, the heterogeneity of IgG₄ molecules is not apparent following gel filtration of purified IgG₄ protein. However, when purified IgG₄ protein is separated by denaturing polyacrylamide gel electrophoresis (SDS-PAGE) under non-reducing conditions, two distinct protein species can be identified. One migrated in the 150 kD size range, consistent with the size of the tetrameric molecule, while the other migrates around the 80 kD size range, which is consistent with the size of a “half immunoglobulin” that contains one heavy chain subunit and one light chain subunit (King et al. (1992), Biochem J 281:317-23).

### SUMMARY OF THE INVENTION

The present invention is based, in part, on the discovery that while several denaturing conditions can trigger the dissociation of “immunoglobulin half antibodies,” most of those conditions cause aggregation and irreversible denaturation and are not easily applicable for the separation of the 80 kD and 150 kD species for biotherapeutics. Dissociation can also be achieved by acidification when the careful choice of conditions makes the dissociation controlled. The current invention provides methodology allowing for the controlled separation of immunoglobulin half antibodies from “immunoglobulin whole antibodies.” While preserving biological activity.

The production of IgG₄ antibodies results in the formation of a mixture of whole and half antibodies. Whole antibodies form a tetramer through inter-heavy chain disulfide bonds in the hinge regions of the heavy chains. Half antibodies, on the other hand, lack these inter-heavy chain disulfide bonds. Nevertheless, it has been found that half antibodies non-covalently interact so as to form tetramers despite the lack of inter-heavy chain disulfide bonds. Due to this non-covalent interaction between half antibodies, their physical properties are highly similar to those of whole antibodies, making it difficult to separate half antibodies from whole antibodies under non-denaturing conditions. The current invention provides methodology to overcome this difficulty with separation.

The present invention is also based, in part, on the discovery that dissociated half antibodies can be chromatographically separated from whole antibodies. Thus, the invention features methods for separating immunoglobulin half antibodies from immunoglobulin whole antibodies, as well as purified immunoglobulin half antibody preparations and purified immunoglobulin whole antibody preparations.

Accordingly, in one aspect, the invention features a method for separating half antibodies from whole antibodies, wherein the half antibodies and the whole antibodies are of the same isotype. The method comprises:

obtaining a sample that contains a mixture of half antibodies and whole antibodies of the same isotype;

reducing the pH of the sample such that the half antibodies dissociate from one another to form a resulting solution; and

applying the resulting solution to a column that differentially retards the mobility of the half antibodies and whole antibodies.

In preferred embodiments, the antibodies are immunoglobulin molecules, e.g., IgG₁, IgG₂, IgG₃, or IgG₄ molecules. Preferably, the antibodies are IgG₄ molecules. In other embodiments, the antibodies are IgA₁ and IgA₂, IgD, IgE, or IgM molecules.

In some embodiments, the antibodies are naturally occurring antibodies, e.g., antibodies produced in a mammal, e.g., mouse monoclonal antibodies or human antibodies. In other embodiments, the antibodies are modified, e.g., recombinant antibodies, e.g., chimeric antibodies, humanized antibodies, or antibody fragments, e.g., F(ab)₂ fragments. In still other embodiments, the antibodies have been modified, e.g., with respect to their affinity and specificity for a particular ligand, e.g., by phage display techniques. The antibodies can be modified, e.g., in the constant or variable region of the light or heavy chain. For example, the antibodies can be modified, e.g., by deletion, insertion, or substitution, at one or more amino acid residues present within one or more CDR and/or framework portion of the variable region of the antibodies, and/or one or more amino acid residues present within the constant regions of the antibodies. The methods of production include in the milk or other bodily fluid of transgenic mammals, in particular ungulates. Most preferably in caprines or bovines.

Other features and advantages of the invention will be apparent from the following detailed description and from the claims.

### BRIEF DESCRIPTION OF THE DRAWINGS

FIG. 1 Shows a flow chart of IgG4 purification and enrichment of the 150 kD species.

FIG. 2A Kinetic Study of IgG4 Dissociation (using various concentrations of citrate).

FIG. 2B Kinetic Study of IgG4 Dissociation (using various concentrations of citrate).

FIG. 2C Kinetic Study of IgG4 Dissociation (using various concentrations of citrate).

FIG. 3A Kinetic Study of IgG4 Dissociation Using pH 3.0 and 100 mM Glycine.

FIG. 3B Kinetic Study of IgG4 Dissociation Using pH 3.0 and 200 mM Glycine.

FIG. 3C Kinetic Study of IgG4 Dissociation Using pH 3.5 and 100 mM Glycine.

FIG. 3D Kinetic Study of IgG4 Dissociation Using pH 3.5 and 200 mM Glycine.

FIG. 4 Kinetic Study of the Antibody Dissociation followed by Size-Exclusion Chromatography.

FIG. 5 Separation of 80 kD and 150 kD Species of IgG4 r-Mab. (Stability of the purified material was tested up to three months. There was no aggregation or degradation detected.)

FIG. 6 Isoelectrofocusing Analysis Of The Cation Exchange Chromatographic Fractions.

FIG. 7 N-Linked Oligosaccharide Profiles of IgG4 CEX 2/5/02 Fractions.

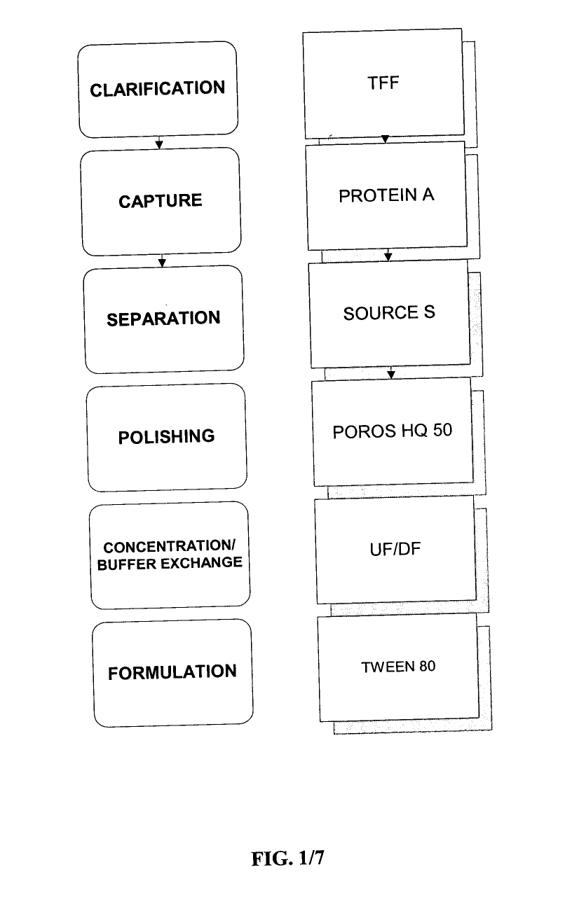

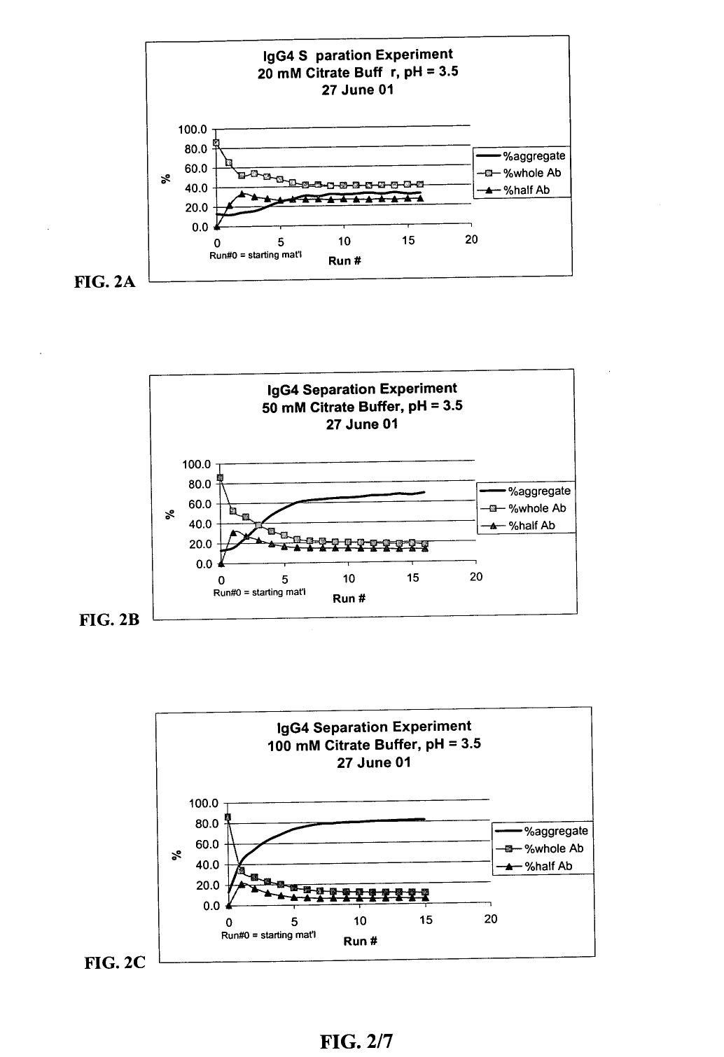

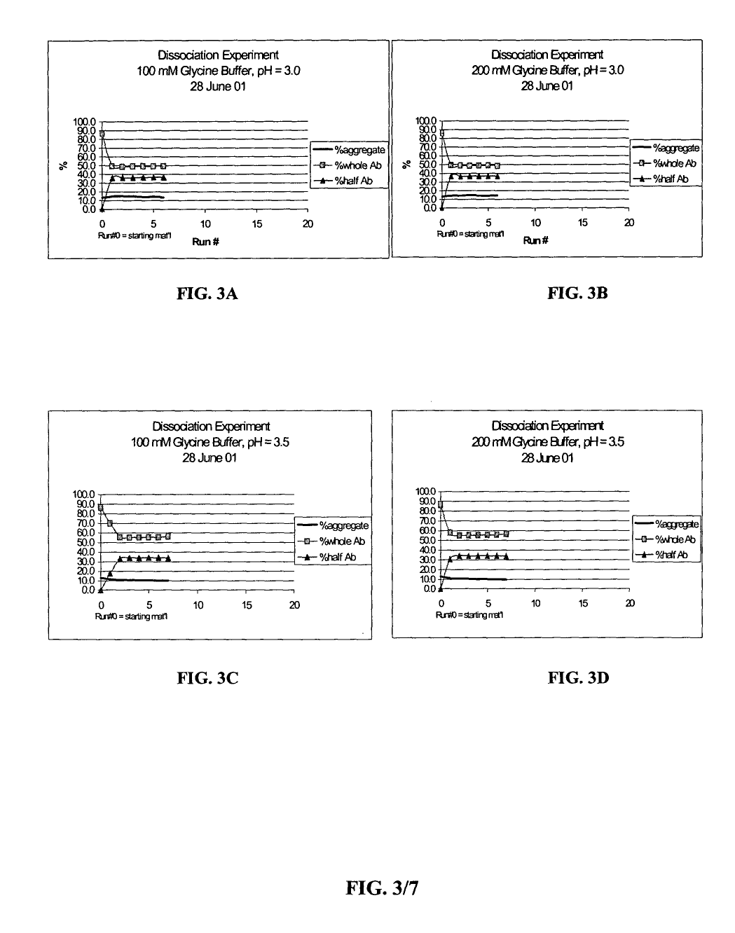

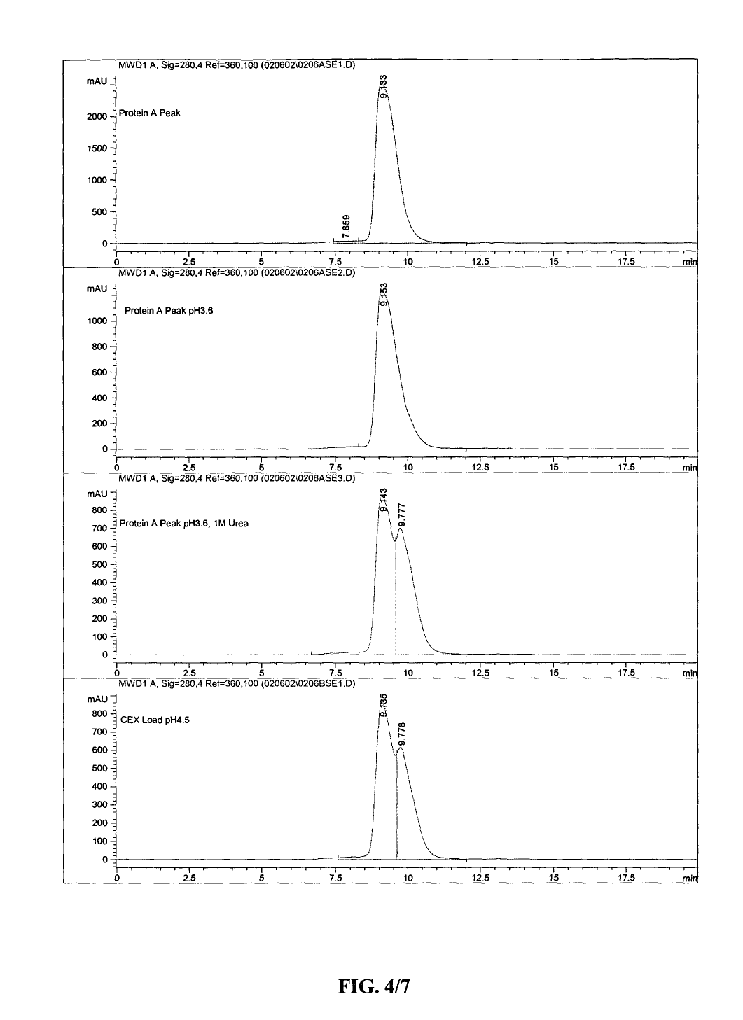

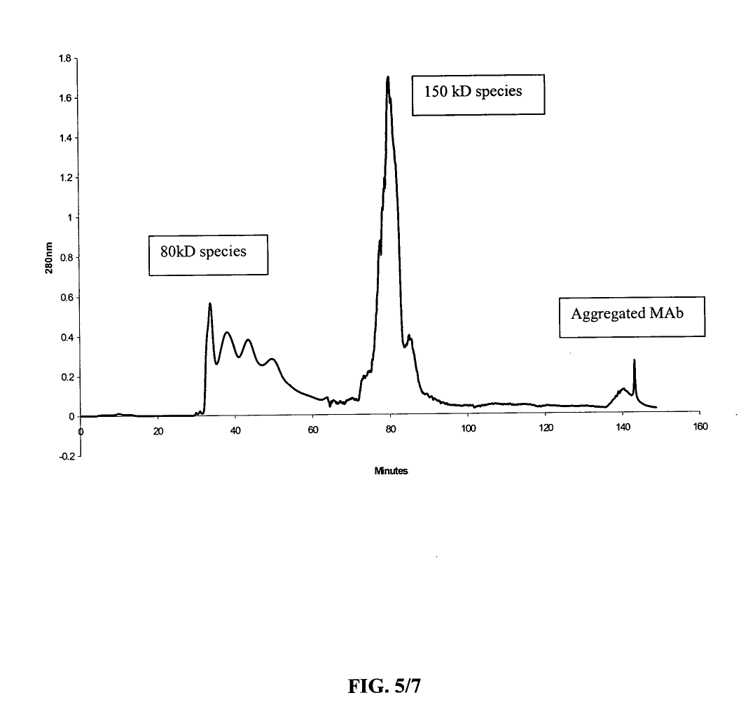

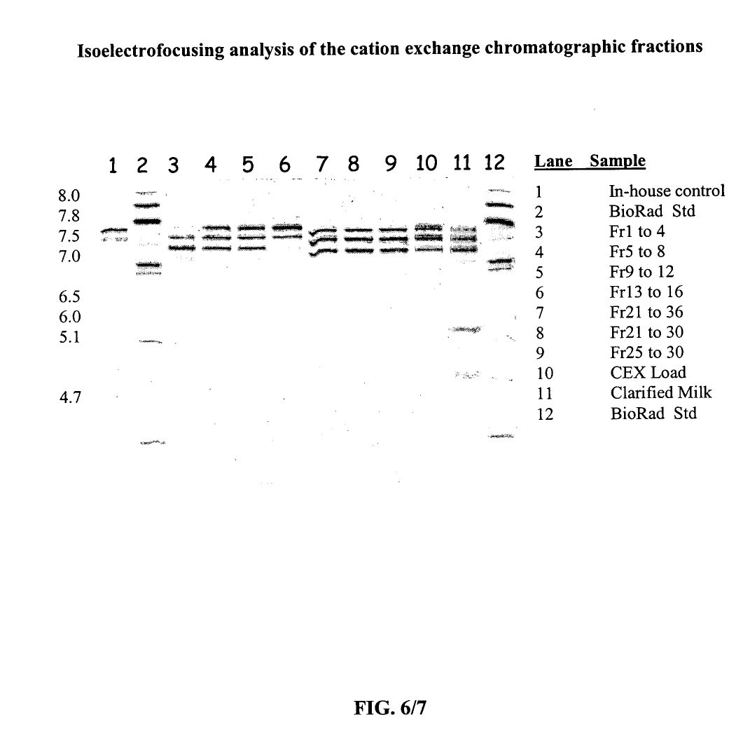

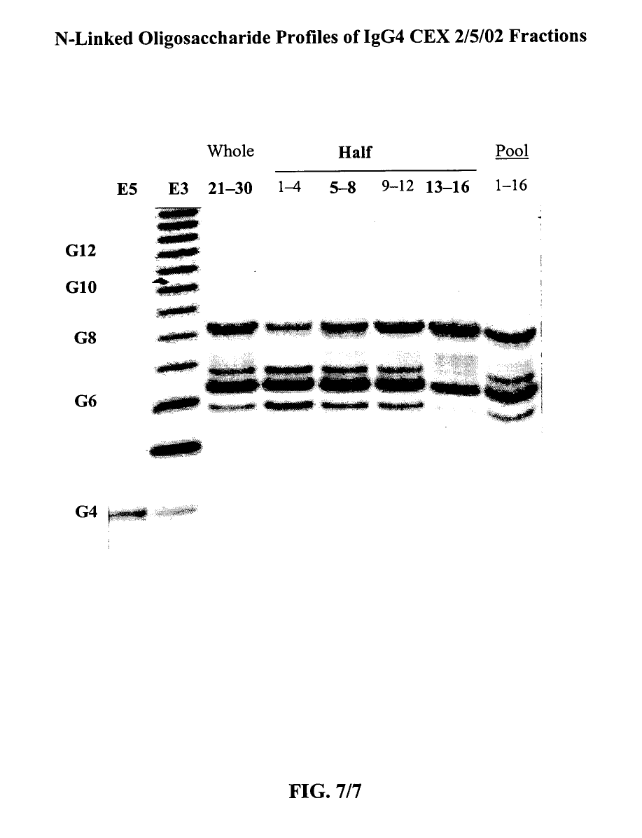

### DETAILED DESCRIPTION

Explanation of Terms:

Ion-Exchange Chromatography:

Proteins are made up of twenty common amino acids. Some of these amino acids possess side groups (“R” groups) which are either positively or negatively charged. A comparison of the overall number of positive and negative charges will give a clue as to the nature of the protein. If the protein has more positive charges than negative charges, it is said to be a basic protein. If the negative charges are greater than the positive charges, the protein is acidic. When the protein contains a predominance of ionic charges, it can be bound to a support that carries the opposite charge. A basic protein, which is positively charged, will bind to a support which is negatively charged. An acidic protein, which is negatively charged, will bind to a positive support. The use of ion-exchange chromatography, then, allows molecules to be separated based upon their charge. Families of molecules (acidics, basics and neutrals) can be easily separated by this technique. This is perhaps the most frequently used chromatographic technique used for protein purification.

Hydrophobic Interaction Chromatography (“HIC”)

HIC allows a much greater selectivity than is observed for ion-exchange chromatography. These hydrophobic amino acids can bind on a support which contains immobilized hydrophobic groups. It should be noted that these HIC supports work by a “clustering” effect; no covalent or ionic bonds are formed or shared when these molecules associate.

Gel-Filtration Chromatography

This technique separates proteins based on size and shape. The support for gel-filtration chromatography are beads which contain holes, called “pores,” of given sizes. Larger molecules, which can't penetrate the pores, move around the beads and migrate through the spaces which separate the beads faster than the smaller molecules, which may penetrate the pores.

Affinity Chromatography

This technique that allows a one-step purification of the target molecule.

This technique is useful for the purification of any protein, provided that a specific ligand is available.

The present invention relates to a system for an improving the separation of whole and half antibodies. As used herein, the terms “Ig” or “antibody” refer to an immunoglobulin molecule, such as an IgA, IgD, IgE, IgG, or IgM molecule or any subclass thereof, e.g., IgG1, IgG2, IgG3, and IgG4.

As used herein, the terms “whole Ig” or “whole antibody” refer to an immunoglobulin molecule, such as an IgA₁ and IgA₂, IgD, IgE, IgG, or IgM molecule or any subclass thereof, that consists of two light chain immunoglobulin subunits and two heavy chain immunoglobulin subunits, wherein the two heavy chain immunoglobulin subunits are covalently bound to one another by one or more disulfide bonds.

A used herein, the terms “half Ig” or “half antibody” refer to an immunoglobulin molecule, such as an IgA, IgD, IgE, IgG, or IgM molecule or any subclass thereof, that consists of either: 1) one light chain immunoglobulin subunit and one heavy chain immunoglobulin subunit; or 2) two light chain immunoglobulin subunits and two heavy chain immunoglobulin subunits, wherein the heavy chain subunits are not covalently bound to one another by disulfide bonds.

As used herein, the term “isotype”, when used to describe an antibody, refers to a particular class and subclass of antibody, e.g., an IgG₄ isotype.

As used herein, the phrase “differentially retards the mobility” refers to a process involving at least two proteins, wherein the proteins are being applied to a column and the time that it takes for one protein to enter and exit the column is, on average, different from the time that it takes the other protein to enter and exit the column.

As used herein, the phrase “interacts with”, as used to describe the interaction of a protein and a column, refers to a process wherein the mobility of the protein is altered by the column. Alterations in the mobility of a protein can result from: transient molecular interactions between the protein and column, e.g., involving van der Waals forces and/or dipole-dipole interactions; stable molecular interactions between the protein and column, e.g., involving van der Waals forces or dipole-dipole interactions; or effects that the column has upon the effective column volume that proteins of different sizes experience as they pass through the column.

As used herein, the phrase “transient molecular interactions” refers to molecular binding interactions that are formed and broken with a half-life of less than one second or are reversible. It is also important to note that with regard with the current invention that the material can be eluted from the column.

As used herein, the phrase “stable molecular interactions” refers to molecular binding interactions that are formed and broken with a half-life equal to or greater than one second.

As used herein, the phrases “is retained by” or “binds”, as used to describe the interaction between a protein and a column, refer to an interaction of sufficient strength and duration such that several column volumes of a suitable wash buffer can be applied to (i.e., passed through) the column without more than 10% of the protein eluting from the column in the wash buffer. Preferably, when a protein is retained by or binds to a column, less than 25%, 10%, 5%, 2%, 1% of the protein will be washed off the column after several column volumes of a suitable wash buffer have been applied to the column.

As used herein, the term “pure,” as applied to a purified preparation of half antibodies, e.g., a chromatographically purified half antibody preparation, refers to a half antibody preparation wherein no more than about 25% or less of the total antibody concentration consists of whole antibodies. Preferably, no more that about 15%, 10%, 5%, 4%, 3%, 2%, 1%, or less of total antibody concentration consists of whole antibodies.

As used herein, the term “pure,” as applied to a purified preparation of whole antibodies, e.g., a chromatographically purified whole antibody preparation, refers to a half antibody preparation wherein no more than about 30% or less of the total antibody concentration consists of half antibodies. Again, it is product specific, but 30 is the highest value reported. Preferably, no more that about 30%, 20%, 15%, 10%, 5%, or less of total antibody concentration consists of half antibodies.

Other terms have their usual definitions, e.g., as they would be defined to one skilled in the art of this invention.

Embodiments of the Current Invention: Alterations of Antibody Structure or Specificity

According to the current invention there are many embodiments that provide for useful modifications of antibody structure. These changes reflect an alteration of the DNA used to manufacture the antibodies in question.

In some embodiments, the antibodies contain a modification of the heavy chain hinge region. For example, the hinge region or a portion thereof has been modified, e.g., by deletion, insertion, or replacement, e.g., with a hinge region or a portion thereof which differs from the hinge region present in a naturally occurring antibody of the same class and subclass.

In some embodiments, the sample is obtained from a mammal, e.g., an ungulate (e.g., a cow, goat, or sheep), pig, rabbit, or mouse. For example, the sample can be obtained from milk, blood (e.g., serum), or a tissue homogenate. In other embodiments, the sample is obtained from a bird, e.g., a chicken, turkey, duck, pheasant, or ostrich. For example, the sample can be obtained from an egg, blood (e.g., serum), or a tissue homogenate. In still other embodiments, the sample is medium that has been used to culture cells, e.g., mammalian cells, avian cells, fish cells, or insect cells. In preferred embodiments, the mammal, bird, or cell that provided the sample is a transgenic mammal, bird, or cell, e.g., a transgenic mammal, bird, or cell which produces an antibody of interest, e.g., an exogenous antibody. In preferred embodiments, the sample is milk obtained from a mammal, e.g., a transgenic mammal which produces an antibody of interest, e.g., an exogenous antibody.

In preferred embodiments, the sample is partially purified prior to reducing the pH of the sample. For example, the sample can be treated to remove non-immunoglobulin proteins, small molecules, and lipids. Such treatments can include chromatography steps, e.g., ion exchange chromatography or affinity chromatography, precipitation steps, and centrifugation steps. For example, milk can be treated to remove casein, cell debris and lipids; eggs can be treated to remove lysozyme; blood can be treated to remove cells and clotting factors, e.g., by initiating clotting; and tissue homogenates and cell culture media can be treated to remove insoluble proteins and cell debris. In some embodiments, the pH of the sample is reduced by adding acid to the sample, e.g., an acidic buffer, e.g., Glycine-HCl, citrate, acetate, formiate buffers or an acidic solution, e.g., a HCl or phosphoric acid solution. In preferred embodiments, the pH of the sample is reduced by adding Glycine-HCl buffer to the sample.

In preferred embodiments, the pH of the sample is reduced until the dissociation is complete. In some embodiments, the pH of the sample is reduced until it is about 4.0, 3.5, or lower, thereby providing a resulting solution wherein most of the half antibodies are dissociated from one another. In preferred embodiments, the pH of the sample is reduced until it is about 3.5.

In some embodiments, the column is an cation exchange column. In other embodiments, the column is a size exclusion column. In still other embodiments, the column is a hydrophobic interaction column.

In still other embodiments, the column is an affinity column. Preferably, the column is a cation exchange column. Source S, S-Sepharose, POROS SH and other high selectivity cation exchangers.

In some embodiments, the column retains (i.e., binds to) the half antibodies present in the resulting solution. In other embodiments, the column retains (i.e., binds to) the whole antibodies present in the resulting solution. In still other embodiments, the column does not retain (i.e., binds to) the half antibodies or the whole antibodies present in the resulting solution, but interacts with (e.g., slows the movement of) the half antibodies, whole antibodies, or both such that the rate at which the half and whole antibodies travel through the column is different. In preferred embodiments, the column retains (i.e., binds to) both the half antibodies and the whole antibodies present in the resulting solution.

In some embodiments, the ion exchange column retains (i.e., binds) most of the antibodies present in the sample. In some embodiments, the ion exchange column retains about 80%, 90%, 95%, 98%, or more of the antibodies present in the sample. In preferred embodiments, the ion exchange column retains (i.e., binds) about 80%, 90%, 95%, 98%, or more of the half antibodies present in the sample. In preferred embodiments, the ion exchange column retains (i.e., binds) about 80%, 90%, 95%, 98%, or more of the Ig whole antibodies present in the sample.

In preferred embodiments, the column binds to the half antibodies under conditions of low pH, e.g., a pH of about 5.0, 4.5, 4.0, 3.5, or lower. In more preferred embodiments, the column binds to the half antibodies under conditions of low pH, but not under conditions of neutral to high pH, e.g., a pH of about 6.5, 7.0, 7.5, or higher.

In preferred embodiments, the column binds to the whole antibodies under conditions of low pH, e.g., a pH of about 5.0, 4.5, 4.0, 3.5, or lower. In more preferred embodiments, the column binds to the whole antibodies under conditions of low pH, as well as condition of neutral to high pH, e.g., a pH of about 6.5, 7.0, 7.5, or higher.

In some embodiments, the method further includes subjecting the column to conditions which selectively elute the half antibodies retained by the column. Such conditions can include, e.g., changing the pH or the ionic strength of the buffer present within the column. In preferred embodiments, the conditions which selectively elute the half antibodies bound to the column comprise adding a buffer to the column such that the pH of the buffer present within the column is increased to a level sufficient to selectively elute the half antibodies.

In some embodiments, the buffer added to the column which increases the pH of the buffer present within the column (i.e., a “high pH buffer”) has a pH of about 4.0 to 8.0. In some embodiments, the high pH buffer includes, e.g., a MES (2-[N-Morpholino]ethanesulfonic acid) HEPES(N-[2-Hydroxyethyl]piperazine-N′[4butanesulfoinic acid), acetate buffer or their mixture. In some embodiments, the high pH buffer Tris buffer (Tris(hydroxymethyl)aminomethane). The list of buffers also includes phosphate buffer, with or without sodium chloride. Some of these buffers may or may not contain ionic or non-ionic detergent like polysorbate 20 or polysorbate 80 or CHAPS or cholate. In preferred embodiments, the high pH buffer has a pH of about 4.0 to 8.0 and includes a HEPES-acetate buffer.

In preferred embodiments, the half antibodies are eluted from the column by increasing the pH of the buffer present within the column to about 6.5, 7.0, 7.5, or more. In preferred embodiments, most of the half antibodies, e.g., 75%, 80%, 85%, 90%, 95%, 98%, or more of the half antibodies, are eluted from the column by increasing the pH of the buffer present within the column to about 6.5, 7.0, 7.5, or more. In preferred embodiments, the half antibodies are eluted from the column by increasing the salt concentration of the buffer present within the column up to 300 mM.

In preferred embodiments, the whole antibodies remain bound to the column when the pH of the buffer present within the column is increase to about 6.5, 7.0, or more. In preferred embodiments, most of the whole antibodies, e.g., 80%, 90%, 95%, 98%, 99%, or more of the whole antibodies, remain bound to the column after the pH of the buffer present within the column is increase to about 6.5, 7.0, or more.

In some embodiments, the buffer being added to the column is added such that the pH of the buffer present within the column increases as a step gradient consisting of one or more steps. In other embodiments, the buffer being added to the column is added such that the pH of the buffer present within the column increases as a linear gradient. In other embodiments, the buffer being added to the column is added such that the pH of the buffer present within the column increases first as a step gradient, e.g., to a pH of about 4.0, 4.5, or 5.0, and then as a linear gradient, e.g., to a pH of about 6.5, 7.0, 7.5, or higher. In still other embodiments, the buffer being added to the column is added such that the pH of the buffer present within the column increases first as a linear gradient, e.g., to a pH of about 4.5, 5.0, or 5.5, and then as a step gradient, e.g., to a pH of about 6.5, 7.0, 7.5, or higher. Preferably, the buffer being added to the column is added such that the pH of the buffer present within the column increases as a step gradient to a pH of about 4.5, and then as a linear gradient to a pH of about 7.0.

In some embodiments, the method further includes subjecting the column to conditions which elute the whole antibodies retained by the column. Such conditions can include, e.g., changing the pH or the ionic strength of the buffer present within the column. In preferred embodiments, the conditions include adding a buffer to the column such that the ionic strength of the buffer present within the column increases in an amount sufficient to elute the whole antibodies. In particularly preferred embodiments, the conditions include adding a buffer to the column such that the pH of the buffer present within the column increases and adding a buffer to the column such that the ionic strength of the buffer present within the column increases, wherein the combination of the increases in pH and ionic strength are sufficient to elute the whole antibodies. In preferred embodiments, the pH and the ionic strength of the buffer present within the column are increased independently. In other embodiments, the pH and the ionic strength of the buffer present within the column are increased simultaneously. In preferred embodiments, the half antibodies are eluted from the column prior to eluting the whole antibodies, and the pH of the buffer present within the column is increased before the ionic strength of the buffer within the column is increased.

In some embodiments, the buffer added to the column which increases the ionic strength of the buffer present within the column (i.e., the “high ionic strength buffer”) includes one or more salts having a high concentration. In some embodiments, the high ionic strength buffer includes at least one salt, e.g., NaCl, KCl, or increased buffer concentration maybe, present at a concentration of at least 5 mM, 100 mM, 150 mM, or more. In preferred embodiments, the high ionic strength buffer includes at least about 50 mM NaCl, or more preferably about 100 mM NaCl. In some embodiments, the high ionic strength buffer further includes other phosphate salts.

In preferred embodiments, the whole antibodies are eluted from the column by: 1) increasing the pH of the buffer present within the column, e.g., to about 5, to 7.0, or more, and 2) increasing the ionic strength of the buffer present within the column, e.g., to the ionic strength of a high ionic strength buffer. In particularly preferred embodiments, the whole antibodies are eluted from the column by increasing the pH of the buffer present within the column to about 7.0 and increasing the ionic strength of the buffer present within the column to the ionic strength of a high ionic strength buffer.

In preferred embodiments, most of the whole antibodies, e.g., 51%, 60%, 70%, 80%, 90%, 95%, 98%, or more of the whole antibodies, are eluted from the column by increasing the pH of the buffer present within the column to about 5.0 to 7.5, or more, and increasing the ionic strength of the buffer present within the column to the ionic strength of a high ionic strength buffer. In preferred embodiments, the eluted whole antibodies are about 70%, 75%, 80%, 85%, 90%, or more pure.

In some embodiments, the high ionic strength buffer added to the column is added such that the ionic strength of the buffer present within the column increases as a step gradient consisting of one or more steps. In other embodiments, the high ionic strength buffer added to the column is added such that the ionic strength of the buffer present within the column increases as a linear gradient. In other embodiments, the high ionic strength buffer added to the column is added such that the ionic strength of the buffer present within the column increases first as a step gradient and then as a linear gradient. In still other embodiments, the high ionic strength buffer added to the column is added such that the ionic strength of the buffer present within the column increases first as a linear gradient and then as a step gradient. In preferred embodiments, the high ionic strength buffer being added to the column is added such that the ionic strength of the buffer present within the column increases to an ionic strength about the same as a 5 mM NaCl solution or higher. In preferred embodiments, the half antibodies are eluted from the column before the whole antibodies are eluted from the column, thereby allowing the half antibodies to be separated from the whole antibodies. In particularly preferred embodiments, most of the half antibodies, e.g., 75%, 80%, 85%, 90%, 95%, 98%, or more of the half antibodies, are eluted from the column before the whole antibodies are eluted from the column, thereby allowing the half antibodies to be separated from the whole antibodies.

In another aspect, the invention features a method for separating half antibodies from whole antibodies, wherein the half antibodies and the whole antibodies are of the same isotype. The method includes:

obtaining a sample that contains a mixture of half antibodies and whole antibodies of the same isotype;

reducing the pH of the sample such that the half antibodies dissociate from one another to form a resulting solution;

applying the resulting solution to an ion exchange column such that both the half antibodies and whole antibodies are retained by the column;

adding a buffer to the column such that the pH of the buffer present within the column increases to a level sufficient to selectively elute the half antibodies; and

adding a buffer to the column such that the ionic strength of the buffer present within the column increases to an amount sufficient to elute the whole antibodies.

In preferred embodiments, the antibodies are immunoglobulin molecules, e.g., IgG1, IgG2, IgG3, or IgG4 molecules. Preferably, the antibodies are IgG4 molecules. In other embodiments, the antibodies are IgA, IgD, IgE, or IgM, molecules.

In some embodiments, the antibodies are naturally occurring antibodies, e.g., antibodies produced in a mammal, e.g., mouse monoclonal antibodies or human antibodies. In other embodiments, the antibodies are modified, e.g., recombinant antibodies, e.g., chimeric antibodies, humanized antibodies, Fc fusion proteins or antibody fragments, e.g., F(ab)₂ fragments. In still other embodiments, the antibodies have been altered, e.g., with respect to their affinity and specificity for a particular ligand, e.g., by phage display techniques. The antibodies can be modified, e.g., in the constant or variable region of the light or heavy chain. For example, the antibodies can be modified, e.g., by deletion, insertion, or substitution, at one or more amino acid residues present within one or more CDR and/or framework portion of the variable regions of the antibodies, and/or one or more amino acid residues present within the constant regions of the antibodies.

In some embodiments, the antibodies contain a modification of the heavy chain hinge region. For example, the hinge region or a portion thereof has been modified, e.g., by deletion, insertion, or replacement, e.g., with a hinge region or a portion thereof which differs from the hinge region present in a naturally occurring antibody of the same class and subclass. For example, an IgG1, IgG2, or IgG3 antibody may contain an IgG4-type hinge region.

In some embodiments, the sample is obtained from a mammal, e.g., an ungulate (e.g., a cow, goat, or sheep), pig, rabbit, or mouse. For example, the sample can be obtained from milk, blood (e.g., serum), or a tissue extract. In other embodiments, the sample is obtained from a bird, e.g., a chicken, turkey, duck, pheasant, or ostrich. For example, the sample can be obtained from an egg, blood (e.g., serum), or a tissue homogenate. In still other embodiments, the sample is cell culture medium that has been used to culture cells, e.g., mammalian cells, avian cells, fish cells, or insect cells. In preferred embodiments, the mammal, bird, or cell that provided the sample is a transgenic mammal, bird, or cell, e.g., a transgenic mammal, bird, or cell which produces an antibody of interest, e.g., an exogenous antibody. In preferred embodiments, the sample is milk obtained from a mammal, e.g., a transgenic mammal which produces an antibody of interest, e.g., an exogenous antibody.

In preferred embodiments, the sample is partially purified prior to reducing the pH of the sample. For example, the sample can be treated to remove non-immunoglobulin proteins, small molecules, and lipids. Such treatments can include chromatography steps, e.g., ion exchange chromatography or affinity chromatography, filtration, precipitation steps, and centrifugation steps. For example, milk can be treated to remove casein and soluble lipids as well as proteins that are non-exogenous immunoglobulins; eggs can be treated to remove lysozyme; blood can be treated to remove cells, e.g., by initiating clotting; and tissue extracts and cell culture media can be treated to remove insoluble proteins and cell debris. In some embodiments, the pH of the sample is reduced by adding acid to the sample, e.g., an acidic buffer, e.g., Glycine-HCl citrate, acetate, formiate buffers or an acidic solution, e.g., a HCl or phosphoric acid solution. In preferred embodiments, the pH of the sample is reduced by adding Glycine-HCl buffer to the sample.

In preferred embodiments, the pH of the sample is reduced until most of the half antibodies are dissociated from one another. In some embodiments, the pH of the sample is reduced until about 60%, 70%, 80%, 90%, 95%, 98%, or more of the half antibodies are dissociated from one another. In some embodiments, the pH of the sample is reduced until it is about 5.0, 4.5, 4.0, 3.5, or lower, thereby providing a resulting solution wherein most of the half antibodies are dissociated from one another. In some embodiments, the pH of the sample is reduced until it is about 2.0 to 4.0. In preferred embodiments, the pH of the sample is reduced until it is about 3.5.

In preferred embodiments, the ion exchange column is a cation exchange column.

In some embodiments, the ion exchange column retains (i.e., binds) most of the antibodies present in the sample. In some embodiments, the ion exchange column retains (i.e., binds) about 51%, 60%, 70%, 80%, 90%, 95%, 98%, or more of the antibodies present in the sample. In preferred embodiments, the ion exchange column retains (i.e., binds) about 51%, 60%, 70%, 80%, 90%, 95%, 98%, or more of the half antibodies present in the sample. In preferred embodiments, the ion exchange column retains (i.e., binds) about 51%, 60%, 70%, 80%, 90%, 95%, 98%, or more of the whole antibodies present in the sample.

In preferred embodiments, the ion exchange column binds to the half antibodies under conditions of low pH, e.g., a pH of about 5.0, 4.5, 4.0, 3.5, or lower. In more preferred embodiments, the ion exchange column binds to the half antibodies under conditions of low pH, but not under conditions of neutral to high pH, e.g., a pH of about 6.5, 7.0, 7.5, or higher.

In preferred embodiments, the ion exchange column binds to the whole antibodies under conditions of low pH, e.g., a pH of about 5.0, 4.5, 4.0, 3.5, or lower. In more preferred embodiments, the ion exchange column binds to the whole antibodies under conditions of low pH, as well as condition of neutral to high pH, e.g., a pH of about 6.5, 7.0, 7.5, or higher.

In some embodiments, the buffer added to the column which increases the pH of the buffer present within the column (i.e., a “high pH buffer”) has a pH of about 4.0 to 8.0. In some embodiments, the high pH buffer includes, e.g., a MES (2-[N-Morpholino]ethanesulfonic acid), HEPES(N-[2-Hydroxyethyl]piperazine-N′[4-butanesulfoinic acid), acetate buffer or their mixture. In some embodiments, the high pH buffer Tris buffer (Tris(hydroxymethyl)aminomethane). The list of buffers also includes phosphate buffer, with or without sodium chloride. Some of these buffers may or may not contain ionic or non-ionic detergent like polysorbate 20 or polysorbate 80 or CHAPS or cholate.

In preferred embodiments, the high pH buffer has a pH of about 4.0 to 8.0 and includes a HEPES-MES-acetate buffer.

In preferred embodiments, the half antibodies are eluted from the column by increasing the pH of the buffer present within the column to about 6.5, 7.0, 7.5, or more. In preferred embodiments, most of the half antibodies, e.g., 75%, 80%, 85%, 90%, 95%, 98%, or more of the half antibodies, are eluted from the column by increasing the pH of the buffer present within the column to about 6.5, 7.0, 7.5, or more. In preferred embodiments, the eluted half antibodies are about 75%, 80%, 85%, 90%, 95%, 98%, 99%, or more whole antibody. That is, when you elute half Ab, the resulting product can be considered a whole antibody or it can be expressed as a level of product with a specific purity level. For example, for the purposes of the current invention we will refer to how much of the Ab is 150 kD in terms of percentages.

In preferred embodiments, the whole antibodies remain bound to the column when the pH of the buffer present within the column is increased to about 6.5, 7.0, 7.5, or more. In preferred embodiments, most of the whole antibodies, e.g., 80%, 90%, 95%, 98%, 99%, or more of the whole antibodies, remain bound to the column after the pH of the buffer present within the column is increased to about 6.5, 7.0, 7.5, or more.

In some embodiments, the high pH buffer added to the column is added such that the pH of the buffer present within the column increases as a step gradient consisting of one or more steps. In other embodiments, the high pH buffer added to the column is added such that the pH of the buffer present within the column increases as a linear gradient. In other embodiments, the high pH buffer added to the column is added such that the pH of the buffer present within the column increases first as a step gradient, e.g., to a pH of about 4.0, 4.5, or 5.0, and then as a linear gradient, e.g., to a pH of about 6.5, 7.0, 7.5, or higher. In still other embodiments, the buffer being added to the column is added such that the pH of the buffer present within the column increases first as a linear gradient, e.g., to a pH of about 4.5, 5.0, or 5.5, and then as a step gradient, e.g., to a pH of about 6.5, 7.0, 7.5, or higher. In preferred embodiments, the buffer being added to the column is added such that the pH of the buffer present within the column increases as a step gradient to a pH of about 4.5, and then as a linear gradient to a pH of about 7.0. We should also note that linear gradient notation can also be used.

In some embodiments, the buffer added to the column which increases the ionic strength of the buffer present within the column (i.e., the “high ionic strength buffer”) includes one or more salts having a high concentration. In some embodiments, the high ionic strength buffer includes at least one salt, e.g., NaCl, KCl, or increased buffer concentration maybe , present at a concentration of at least 5 mM, 100 mM, 150 mM, or more In preferred embodiments, the high ionic strength buffer includes at least about 5 mM NaCl, more preferably about 100 mM NaCl.or more. In some embodiments, the high ionic strength buffer further comprisesMES (2-[N-Morpholino]ethanesulfonic acid), HEPES(N-[2-Hydroxyethyl]piperazine-N′[4-butanesulfoinic acid), acetate buffer or their mixture. In some embodiments, the high pH buffer may be Tris buffer (Tris(hydroxymethyl)aminomethane). The list of buffers also includes phosphate buffer, with or without sodium chloride. Some of these buffers may or may not contain ionic or non-ionic detergent like polysorbate 20 or polysorbate 80 or CHAPS or cholate.

In some embodiments, the whole antibodies are eluted from the column by increasing both the pH and the ionic strength of the buffer present within the column. In preferred embodiments, the whole antibodies are eluted from the column by: 1) increasing the pH of the buffer present within the column, e.g., to about 6.5, 7.0, 7.5, or more, and 2) increasing the ionic strength of the buffer present within the column, e.g., to the ionic strength of a high ionic strength buffer. In particularly preferred embodiments, the whole antibodies are eluted from the column by increasing the pH of the buffer present within the column to about 7.0 and increasing the ionic strength of the buffer present within the column to the ionic strength of a high ionic strength buffer.

In preferred embodiments, most of the whole antibodies, e.g., 51%, 60%, 70%, 80%, 90%, 95%, 98%, or more of the whole antibodies, are eluted from the column by increasing the pH of the buffer present within the column, e.g., to a pH of about 6.5, 7.0, 7.5, or more, and increasing the ionic strength of the buffer present within the column, e.g., to the value of a high ionic strength buffer. In preferred embodiments, the eluted whole antibodies are about 70%, 75%, 80%, 85%, 90%, or more pure.

In some embodiments, the high ionic strength buffer added to the column is added such that the ionic strength of the buffer present within the column increases as a step gradient consisting of one or more steps. In other embodiments, the high ionic strength buffer added to the column is added such that the ionic strength of the buffer present within the column increases as a linear gradient. In other embodiments, the high ionic strength buffer added to the column is added such that the ionic strength of the buffer present within the column increases first as a step gradient and then as a linear gradient. In still other embodiments, the high ionic strength buffer added to the column is added such that the ionic strength of the buffer present within the column increases first as a linear gradient and then as a step gradient. In preferred embodiments, the high ionic strength buffer being added to the column is added such that the ionic strength of the buffer present within the column increases to an ionic strength about the same as a 5 mM NaCl solution.

In preferred embodiments, the half antibodies are eluted from the column before the whole antibodies are eluted from the column, thereby allowing the half antibodies to be separated from the whole antibodies. In particularly preferred embodiments, most of the half antibodies, e.g., 75%, 80%, 85%, 90%, 95%, 98%, or more of the half antibodies, are eluted from the column before the whole antibodies are eluted from the column, thereby allowing the half antibodies to be separated from the whole antibodies.

In another aspect, the invention features a purified half antibody preparation obtained by a method described herein.

In preferred embodiments, the purified half antibody preparation includes gamma immunoglobulin containing molecules, e.g., IgG₁, IgG₂, IgG₃, or IgG₄ half antibodies. Preferably, the purified half antibody preparation includes IgG₄ half antibodies. In other embodiments, the purified half antibody preparation may include IgA, IgD, IgE, or IgM, half antibodies.

In some embodiments, the purified half antibody preparation includes naturally occurring half antibodies, e.g., half antibodies produced in a mammal, e.g., mouse monoclonal half antibodies or human half antibodies. In other embodiments, the purified half antibody preparation includes modified half antibodies, e.g., recombinant half antibodies, e.g., chimeric half antibodies, humanized half antibodies, Fc fusion proteins where the variable region is replaced by an other polypeptide or half antibody fragments, e.g., half antibodies obtained from F(ab)₂ fragments In still other embodiments, the purified half antibody preparation includes half antibodies that have been altered, e.g., with respect to their affinity and specificity for a particular ligand, e.g., by phage display techniques. The half antibodies can be modified, e.g., in the constant or variable region of the light or heavy chain. For example, the half antibodies can be modified, e.g., by deletion, insertion, or substitution, at one or more amino acid residues present within one or more CDR and/or framework portion of the variable region of the half antibodies, and/or one or more amino acid residues present within the constant regions of the half antibodies.

In some embodiments, the purified half antibody preparation includes half antibodies that contain a modification of their heavy chain hinge region. For example, the hinge region or a portion thereof has been modified, e.g., by deletion, insertion, or replacement, e.g., with a hinge region or a portion thereof which differs from the hinge region present in a naturally occurring antibody of the same class and subclass. For example, an IgG1, IgG2, or IgG3 half antibody may contain an IgG4type hinge region.

In some embodiments, the half antibodies present in the purified half antibody preparation constitute 80%, 85%, 90%, 95%, 98%, 99%, or more of the total antibodies present in the preparation. In preferred embodiments, the half antibodies present in the purified half antibody preparation constitute at least 80% of the total antibodies present in the preparation.

In some embodiments, the purified half antibody preparation contains contaminants, e.g., protein, small molecule, nucleic acid and/or lipid contaminants. Such contaminants can, for example, be a reflection of the sample from which the purified half antibody preparation was obtained and/or the process used to obtain the preparation. For example, if the purified half antibody preparation was obtained from a sample of milk, the preparation may contain contaminant proteins or small molecules typically found milk, e.g., casein, lactose, Calcium phosphate, caseins, α-lactalbumin, β-lactoglobulin, lactoferrin, and/or trace amounts of blood serum proteins endogenous immunoglobulins like endogenous immunoglobulins. If obtained from an egg, it may contain contaminant proteins or small molecules typically found in eggs, e.g., lysozyme, ovalbumin. If obtained from animal sera it may contain contaminant proteins or small molecules typically found in blood, e.g., glucose, cholesterol, hemoglobin, albumin, endogenous antibodies. In a final source or feedstream that may be purified by the methods of the current invention if the preparation was obtained from cell culture medium, it may contain contaminant proteins or small molecules typically found in cell culture medium, e.g., extracellular matrix proteins, penicillin, glucose and other components originated from the cell culture media.

In another aspect, the invention features a purified whole antibody preparation obtained by a method described herein.

In preferred embodiments, the purified whole antibody preparation includes gamma immunoglobulin containing molecules, e.g., IgG1, IgG2, IgG3, or IgG4 whole antibodies. Preferably, the purified whole antibody preparation includes IgG4 whole antibodies. In other embodiments, the purified whole antibody preparation includes IgA, IgD, IgE, or IgM, whole antibodies.

In some embodiments, the purified whole antibody preparation includes naturally occurring whole antibodies, e.g., whole antibodies produced in a mammal, e.g., mouse or rat monoclonal whole antibodies or human whole antibodies. In other embodiments, the purified whole antibody preparation includes modified whole antibodies, e.g., recombinant whole antibodies, e.g., chimeric whole antibodies, humanized whole antibodies, Fc fusion proteins or it's fragments or whole antibody fragments, e.g., F(ab)₂ fragments. In still other embodiments, the purified whole antibody preparation includes whole antibodies that have been altered, e.g., with respect to their affinity and specificity for a particular ligand, e.g., by phage display techniques. The whole antibodies can be modified, e.g., in the constant or variable region of the light or heavy chain. For example, the whole antibodies can be modified, e.g., by deletion, insertion, or substitution, at one or more amino acid residues present within one or more CDR and/or framework portion of the variable region of the whole antibodies, and/or one or more amino acid residues present within the constant regions of the whole antibodies.

In some embodiments, the purified whole antibody preparation includes whole antibodies that contain a modification of their heavy chain hinge region. For example, the hinge region or a portion thereof has been modified, e.g., by deletion, insertion, or replacement, e.g., with a hinge region or a portion thereof which differs from the hinge region present in a naturally occurring antibody of the same class and subclass. For example, an IgG1, IgG2, or IgG3 whole antibody may contain an IgG4-type hinge region.

In some embodiments, the whole antibodies present in the purified whole antibody preparation constitute 60%, 70%, 80%, 85%, 90%, or more of the total antibodies present in the preparation. In preferred embodiments, the whole antibodies present in the purified whole antibody preparation constitute at least 90% of the total antibodies present in the preparation.

In some embodiments, the whole antibody preparation contains both whole antibodies and half antibodies. In preferred embodiments, the whole antibodies constitute at least 80%, 85%, 90%, 95%, or more of the total amount of antibodies present in such a preparation. In particularly preferred embodiments, the whole antibodies constitute at least 90% or more of the total amount of antibodies present in such a preparation.

In another aspect, the invention features a purified half antibody preparation.

In preferred embodiments, the purified half antibody preparation includes gamma immunoglobulin containing molecules, e.g., IgG1, IgG2, IgG3, or IgG4 half antibodies. In particularly preferred embodiments, the purified half antibody preparation includes IgG4 half antibodies. In other embodiments, the purified half antibody preparation includes IgA, IgD, IgE, or IgM, half antibodies.

In some embodiments, the purified half antibody preparation includes naturally occurring half antibodies, e.g., half antibodies produced in a mammal, e.g., mouse monoclonal half antibodies or human half antibodies. In other embodiments, the purified half antibody preparation includes modified half antibodies, e.g., recombinant half antibodies, e.g., chimeric half antibodies, humanized half antibodies, or half antibody fragments, e.g., half antibodies obtained from F(ab)₂ fragments. In still other embodiments, the purified half antibody preparation includes half antibodies that have been altered, e.g., with respect to their affinity and specificity for a particular ligand, e.g., by phage display techniques. The half antibodies can be modified, e.g., in the constant or variable region of the light or heavy chain. For example, the half antibodies can be modified, e.g., by deletion, insertion, or substitution, at one or more amino acid residues present within one or more CDR and/or framework portion of the variable region of the half antibodies, and/or one or more amino acid residues present within the constant regions of the half antibodies.

In some embodiments, the purified half antibody preparation includes half antibodies that contain a modification of their heavy chain hinge region. For example, the hinge region or a portion thereof has been modified, e.g., by deletion, insertion, or replacement, e.g., with a hinge region or a portion thereof which differs from the hinge region present in a naturally occurring antibody of the same class and subclass. For example, an IgG1, IgG2, or IgG3 half antibody may contain an IgG4-type hinge region.

In some embodiments, the half antibodies present in the purified half antibody preparation constitute 80%, 85%, 90%, 95%, 98%, 99%, or more of the total antibodies present in the preparation. In preferred embodiments, the half antibodies present in the purified half antibody preparation constitute at least 99% of the total antibodies present in the preparation.

In some embodiments, the purified half antibody preparation contains contaminants, e.g., protein, small molecule, and/or lipid contaminants. Such contaminants can, for example, be a reflection of the sample from which the purified half antibody preparation was obtained and/or the process used to obtain the preparation. For example, if the purified half antibody preparation was obtained from a sample of milk, the preparation may contain contaminant proteins or small molecules typically found milk, e.g., casein, lactose, Calcium phosphate, caseins, α-lactalbumin, β-lactoglobulin, lactoferrin, and/or trace amounts of blood serum proteins endogenous immunoglobulins like endogenous immunoglobulins . . . if the preparation was obtained from an egg, it may contain contaminant proteins or small molecules typically found in eggs, e.g., lysozyme, . . . if the preparation was obtained from serum, it may contain contaminant proteins or small molecules typically found in blood, e.g., glucose, cholesterol, hemoglobin, albumin, endogenous antibodies . . . or if the preparation was obtained from cell culture medium, it may contain contaminant proteins or small molecules typically found in cell culture medium, e.g., extracellular matrix proteins, penicillin, glucose and other components originated from the cell culture media.

In another aspect, the invention features a purified antibody preparation wherein the preparation includes the separation of half antibodies and whole antibodies.

In preferred embodiments, the purified antibody preparation includes immunoglobulin containing molecules, e.g., IgG₁, IgG₂, IgG3, or IgG₄ whole and half antibodies. In particularly preferred embodiments, the purified antibody preparation includes IgG₄ whole and half antibodies. In other embodiments, the purified antibody preparation includes IgA, IgD, IgE, or IgM, whole and half antibodies. These antibodies may be those of any mammal but they are preferably fully human or humanized antibodies.

In some embodiments, the purified antibody preparation includes naturally occurring antibodies, e.g., antibodies produced in a mammal, e.g., mouse or rat monoclonal antibodies or human antibodies. In other embodiments, the purified antibody preparation includes modified antibodies, e.g., recombinant antibodies, e.g., chimeric antibodies, transgenic antibodies, humanized antibodies, or antibody fragments, e.g., F(ab) and F(ab)₂ fragments. In still other embodiments, the purified antibody preparation includes antibodies that have been altered, e.g., with respect to their affinity and specificity for a particular ligand, e.g., by phage display techniques. The antibodies can be modified, e.g., in the constant or variable region of the light or heavy chain. For example, the antibodies can be modified, e.g., by deletion, insertion, or substitution, at one or more amino acid residues present within one or more CDR and/or framework portion of the variable region of the antibodies, and/or one or more amino acid residues present within the constant regions of the antibodies.

In some embodiments, the purified antibody preparation includes antibodies that contain a modification of their heavy chain hinge region. For example, the hinge region or a portion thereof has been modified, e.g., by deletion, insertion, or replacement, e.g., with a hinge region or a portion thereof which differs from the hinge region present in a naturally occurring antibody of the same class and subclass. For example, an IgG1, IgG2, or IgG3 antibody may contain an IgG4-type hinge region.

In some embodiments, the whole antibodies present in the purified antibody preparation constitute 60%, 70%, 80%, 85%, 90%, or more of the total antibodies present in the preparation. In preferred embodiments, the whole antibodies present in the purified antibody preparation constitute at least 70% of the total antibodies present in the preparation.

In some embodiments, the purified antibody preparation contains contaminants, e.g., protein, nucleic acid, small molecule, and/or lipid contaminants. Such contaminants can, for example, be a reflection of the sample from which the purified antibody preparation was obtained and/or the process used to obtain the preparation. For example, if the purified antibody preparation was obtained from a sample of milk, the preparation may contain contaminant proteins or small molecules typically found milk, e.g., casein, lactose, calcium phosphate, caseins, α-lactalbumin, β-lactoglobulin, lactoferrin, and/or trace amounts of blood serum proteins endogenous immunoglobulins like endogenous immunoglobulins. If the preparation was obtained from an egg, it may contain contaminant proteins or small molecules typically found in eggs, e.g., lysozyme, ovalbumin. If the preparation was obtained from serum, it may contain contaminant proteins or small molecules typically found in blood, e.g., glucose, cholesterol, hemoglobin, albumin, endogenous antibodies. Finally if the preparation was obtained from cell culture medium, it may contain contaminant proteins or small molecules typically found in cell culture medium, e.g., extracellular matrix proteins, penicillin, glucose and other components originated from the cell culture media or bioreactor container.

### EXAMPLE 1

### Optimized Separation of Two Forms of hIgG₄

1. Isolation of rhIgG4 from goat milk.

2. Separation of 80 kD and 150 kD species

3. Formulation

1. Isolation of rhIG4 from Goat Milk.

Goat milk was clarified by dual tangential flow filtration, the clarified milk was applied to POROS Protein A 50 column at 10 mg/mL loading capacity. Linear velocity 120 cm/hr.

Elution was performed with 0.2 M Glycine-HCl pH 3.5.

pH of the eluted protein was adjusted to 3.6

Antibody was incubated at ambient temperature for one hour.

2. Separation of 80 kD and 150 kD Species (Half and Whole IgG₄)

The pH was adjusted to 4.5 and the material was loaded onto a Source S column

Loading capacity 9 mg/mL

Linear velocity 120 cm/hr

Elution of 80 kD species (half antibody) was performed by using a pH gradient form pH 4.5 to pH 7.3

Elution of the 150 kD species (whole antibody) was performed by applying a small increase of NaCl concentration (0-10 mM).

3. Formulation

Whole and half antibody fractions were pooled, concentrated and buffer exchanged

TABLE 1
Stability Study of the Unfractionated, Whole Antibody Enriched and
Half Antibody Enriched Materials
(Aggregation Analyzed by Size-Exclusion Chromatography)

| Sample | % Monomer |
|---|---|
| 150 kD IgG4 at 12 weeks | 98.8 |
| 150 kD IgG4 at 9 weeks | 99.6 |
| 150 kD IgG4 at 8 weeks | 99.6 |
| PA Purified IgG4 7 weeks | 99.2 |
| 80 kD IgG4 at 6 weeks | 99.8 |

TABLE 2
Stability Study of the Whole Antibody Enriched Material
(SDS-PAGE under non-reducing conditions)

| Time Point | 150 kD MAb Content (%) |
|---|---|
| Time 0 | 82 |
| 1 week | 82 |
| 2 weeks | 81 |
| 3 weeks | 81 |
| 4 weeks | 81 |
| 5 weeks | 81 |
| 7 weeks | 82 |
| 8 weeks | 82 |

Antibody Production In Transgenic Animals

Immunoglobulins are heteropolymeric proteins that are normally synthesized, modified, assembled, and secreted from circulating B lymphocytes. Using recombinant DNA technology, it is possible to program cells other than B-lymphocytes to express immunoglobulin genes. The difficulties encountered in this effort stem from several factors: 1) Both heavy and light chains of immunoglobulins must be co-expressed at appropriate levels; 2) Nascent immunoglobulin polypeptides undergo a variety of co- and post-translational modifications that may not occur with sufficient fidelity or efficiency in heterologous cells; 3) Immunoglobulins require accessory chaperone proteins for their assembly; 4) The synthetic and secretary capacity of the cell may be inadequate to secrete large amounts of heterologous proteins; and 5) The secreted immunoglobulins may be unstable in the extracellular milieu of a foreign cell.

Because immunoglobulins have many therapeutic, diagnostic and industrial applications, there is a need in the art for expression systems in which these proteins can be reproducibly manufactured at a high level, in a functional configuration, and in a form that allows them to be easily harvested and purified. The development of transgenic animal technology has raised the possibility of using large animals as genetically programmed protein factories. P.C.T. application WO 90/04036 (published Apr. 19, 1990) discloses the use of transgenic technology for immunoglobulin expression. WO 92/03918 (Mar. 19, 1992). and WO 93/12227 (Jun. 24, 1993) teach the introduction of unrearranged immunoglobulin genes into the germline of transgenic animals. The use of intact immunoglobulin genes (including their respective promoter regions) will result in their expression in lymphocytes and secretion into the bloodstream of the host animal; this necessitates a strategy for suppressing the expression of the host's endogenous immunoglobulins, and raises the problem of purifying the immunoglobulins from serum, which contains many other proteins, including proteolytic enzymes. Furthermore, if the transgenic approach is chosen, heavy and light chain genes must both be incorporated into the host genome, in a manner that enables their comcomittant expression.

The present invention pertains to a method for the production of monoclonal antibodies that are excreted into the milk of transgenic animals and the method for production of such animals. This is achieved by engineering DNA constructs in which DNA segments encoding specific paired immunoglobulin heavy and light chains are cloned downstream of a promoter sequence that is preferentially expressed in mammary epithelial cells. The recombinant DNAs containing the promoter-linked heavy and light chain genes are then coinjected into preimplantation embryos. The progeny are screened for the presence of both transgenes. Representative females from these lines are then milked, and the milk is analyzed for the presence of the monoclonal antibody. In order for the antibody to be present, both heavy and light chain genes must be expressed concurrently in the same cell. The antibodies may be purified from the milk, or the milk itself, comprising the immunoglobulins, may be used to deliver the antibodies to a recipient.

The immunoglobulin genes useful in the present invention may be obtained from natural sources e.g. individual B cell clones or hybridomas derived therefrom. Alternately, they may comprise synthetic single-chain antibodies in which the light and heavy variable regions are expressed as part of a single polypeptide. Furthermore, recombinant antibody genes may be used that have been predictively altered by nucleotide substitutions that do or do not change the amino acid sequence, by addition or deletion of sequences, or by creation of hybrid genes in which different regions of the polypeptide are derived from different sources. Antibody genes by their nature are extremely diverse, and thus naturally tolerate a great deal of variation. It will be appreciated by those skilled in the art that the only limitation for producing an antibody by the method of the present invention is that it must assemble into a functional configuration and be secreted in a stable form into the milk.

The transcriptional promoters useful in practicing the present invention are those promoters that are preferentially activated in mammary epithelial cells, including promoters that control the genes encoding milk proteins such as caseins, beta lactoglobulin (Clark et al., (1989) Bio/Technology 7: 487-492), whey acid protein (Gordon et al., (1987) Bio/Technology 5: 1183-1187), and lactalbumin (Soulier et al., (1992) FEBS Letts. 297: 13). Casein promoters may be derived from the alpha, beta, or kappa casein genes of any mammalian species; a preferred promoter is derived from the goat beta-casein gene (DiTullio, (1992) Bio/Technology 10:74-77).

For use in the present invention, the following methodology may be used: a unique XhoI restriction site is introduced at the 3′ terminus of the promoter sequence to allow the routine insertion of immunoglobulin coding sequences. Preferably, the inserted immunoglobulin gene is flanked on its 3′ side by cognate genomic sequences from a mammary-specific gene, to provide a polyadenylation site and transcript-stabilizing sequences. Transcription of the construct in vivo results in the production of a stable mRNA containing casein-derived 5′ untranslated sequences upstream of the translational initiator codon of the immunoglobulin gene and 3′ untranslated sequences downstream of the translational termination codon of the immunoglobulin gene. Finally, the entire cassette (i.e. promoter-immunoglobulin-3′ region) is flanked by restriction sites that enable the promoter-cDNA cassette to be easily excised as a single fragment. This facilitates the removal of unwanted prokaryotic vector-derived DNA sequences prior to injection into fertilized eggs.

The promoter-linked immunoglobulin heavy and light chain DNAs are then introduced into the germ line of a mammal e.g. cow, sheep, goat, mouse, oxen, camel or pig. Mammals are defined herein as all animals, excluding humans, that have mammary glands and produce milk. Mammalian species that produce milk in large amounts over long periods of time are preferred. Typically, the DNA is injected into the pronuclei of fertilized eggs, which are then implanted into the uterus of a recipient female and allowed to gestate. After birth, the putative transgenic animals are tested for the presence of the introduced DNA. This is easily achieved by Southern blot hybridization of DNA extracted from blood cells or other available tissue, using as a probe a segment of the injected gene that shows no cross hybridization with the DNA of the recipient species. Progeny that show evidence of at least one copy of both heavy and light-chain immunoglobulin genes are selected for further analysis.

Transgenic females may be tested for immunoglobulin secretion into milk, using any of the immunological techniques that are standard in the art (e.g. Western blot, radioimmunoassay, ELISA). The anti-immunoglobulin antibodies used in this analysis may be polyclonal or monoclonal antibodies that detect isolated heavy or light chains or others that react only with fully assembled (H2L2) immunoglobulins.

The recombinant immunoglobulins are also characterized with respect to their functionality, i.e. binding specificity and affinity for a particular antigen. This is achieved using immunological methods that are standard in the art, such as Scatchard analysis, binding to immobilized antigen, etc. The stability characteristics of an immunoglobulin in the milk of a given species are also assayed, by applying the above-described detection methods to milk that has been incubated for increasing times after recovery from the animal.

The immunoglobulins produced by the methods of the present invention may be purified from milk, using adsorption to immobilized Protein G, column chromatography, and other methods known to those of ordinary skill in the art of antibody purification.

The level of production of recombinant immunoglobulins in an individual transgenic mammal is primarily determined by the site and manner of integration of the transgene after injection into the fertilized egg. Thus, transgenic progeny derived from different injected eggs may vary with respect to this parameter. The amount of recombinant immunoglobulin in milk is therefore monitored in representative progeny, and the highest-producing females are preferred.

Those skilled in the art will recognize that the methods of the present invention can be used to optimize the production of natural and synthetic immunoglobulins. The steps of creating a transgenic animal, testing for the presence of both heavy and light-chain genes, assaying the secretion of immunoglobulin into the milk of female progeny, and, finally, assessing the quality of the resulting antibodies, can be repeated sequentially, without undue experimentation, to establish preferred constructs for different applications.

According to the present invention, the nature of the recombinant immunoglobulins and their specific mode of use can vary. In one embodiment, the present invention encompasses high-level expression of antibodies that are harvested and purified from milk and used in purified form. High-level expression is defined herein as the production of about 1 mg/ml of protein. In another embodiment, desirable antibodies are engineered that provide protection to humans against infectious diseases; therapeutic administration is then achieved by drinking the milk. In a still further embodiment, lactating animals are engineered to produce antibodies specifically beneficial to their offspring, which acquire them through suckling. In a still further embodiment, animals produce an antibody that protects the lactating mammal itself against breast pathogens e.g. bacteria that produce mastitis.

The transgenic, recombinant, or chimeric antibodies, (e.g., half-antibodies and/or whole antibodies) produced according to the invention find use in a wide variety of therapeutic procedures, such as in preparation of pharmaceutical compositions for administration to patients or in diagnosis of diseases. For example, transgenically produced antibodies can be useful as anti-arthritis agents or anti-cancer agents as is known in the field.

The application of transgenic technology to the commercial production of recombinant antibodies in the milk of transgenic animals using milk protein specific signal and promoter sequences offers significant advantages over traditional methods of antibody production. These advantages include a reduction in the total amount of required capital expenditures, elimination of the need for capital commitment to build facilities early in the product development life cycle, and lower direct production cost per unit for hard to produce antibodies. Of key importance are the separation techniques made available by the current invention that allow the disassociation of whole antibodies and antibodies that do not have all of their disulfide linkages intact. In fact, transgenic production may represent the only technologically and economically feasible method of commercial production.

The method of the invention demonstrates a strategy that leads to the efficient secretion of normally non-secreted proteins, e.g., antibodies or antibody fragments in the milk of transgenic mammals. It has been demonstrated herein that adding a goat β-casein signal peptide, or β-casein signal peptide and the N-terminal portion of β-casein, to the N-terminal portion of an antibody's DNA transcript is sufficient to secrete these normally cytoplasmic proteins in the milk of transgenic mice and other transgenic animals. Thus, the method of the invention facilitates the reliabe and consistent production of desirable antibodies or fragments thereof in the milk of transgenic mammals.

Milk Specific Promoters

The transcriptional promoters useful in practicing the present invention are those promoters that are preferentially activated in mammary epithelial cells, including promoters that control the genes encoding milk proteins such as caseins, beta lactoglobulin (Clark et al., (1989) Bio/Technology 7: 487-492), whey acid protein (Gorton et al. (1987) Bio/Technology 5: 1183-1187), and lactalbumin (Soulier et al., (1992) FEBS Letts. 297: 3). Casein promoters may be derived from the alpha, beta, gamma or kappa casein genes of any mammalian species; a preferred promoter is derived from the goat beta casein gene (DiTullio, (1992) Bio/Technology 10:74-77). The milk-specific protein promoter or the promoters that are specifically activated in mammary tissue may be derived from either cDNA or genomic sequences. Preferably, they are genomic in origin.

DNA sequence information is available for all of the mammary gland specific genes listed above, in at least one, and often several organisms. See, e.g., Richards et al., J. Biol. Chem. 256, 526-532 (1981) (α-lactalbumin rat); Campbell et al., Nucleic Acids Res. 12, 8685-8697 (1984) (rat WAP); Jones et al., J. Biol. Chem. 260, 7042-7050 (1985) (rat β-casein); Yu-Lee & Rosen, J. Biol. Chem. 258, 10794-10804 (1983) (rat γ-casein); Hall, Biochem. J. 242, 735-742 (1987) (α-lactalbumin human); Stewart, Nucleic Acids Res. 12, 389 (1984) (bovine αs1 and κ casein cDNAs); Gorodetsky et al., Gene 66, 87-96 (1988) (bovine β casein); Alexander et al., Eur. J. Biochem. 178, 395-401 (1988) (bovine κ casein); Brignon et al., FEBS Lett. 188, 48-55 (1977) (bovine αS2 casein); Jamieson et al., Gene 61, 85-90 (1987), Ivanov et al., Biol. Chem. Hoppe-Seyler 369, 425-429 (1988), Alexander et al., Nucleic Acids Res. 17, 6739 (1989) (bovine β lactoglobulin); Vilotte et al., Biochimie 69, 609-620 (1987) (bovine α-lactalbumin). The structure and function of the various milk protein genes are reviewed by Mercier & Vilotte, J. Dairy Sci. 76, 3079-3098 (1993) (incorporated by reference in its entirety for all purposes). To the extent that additional sequence data might be required, sequences flanking the regions already obtained could be readily cloned using the existing sequences as probes. Mammary-gland specific regulatory sequences from different organisms are likewise obtained by screening libraries from such organisms using known cognate nucleotide sequences, or antibodies to cognate proteins as probes.

Signal Sequences

Among the signal sequences that are useful in accordance with this invention are milk-specific signal sequences or other signal sequences which result in the secretion of eukaryotic or prokaryotic proteins. Preferably, the signal sequence is selected from milk-specific signal sequences, i.e., it is from a gene which encodes a product secreted into milk. Most preferably, the milk-specific signal sequence is related to the milk-specific promoter used in the expression system of this invention. The size of the signal sequence is not critical for this invention. All that is required is that the sequence be of a sufficient size to effect secretion of the desired recombinant protein, e.g., in the mammary tissue. For example, signal sequences from genes coding for caseins, e.g., alpha, beta, gamma or kappa caseins, beta lactoglobulin, whey acid protein, and lactalbumin are useful in the present invention. The preferred signal sequence is the goat β-casein signal sequence.

Signal sequences from other secreted proteins, e.g., proteins secreted by liver cells, kidney cell, or pancreatic cells can also be used.

Accordingly, it is to be understood that the embodiments of the invention herein providing for improved methods for the separation of half-antibodies from whole antibodies when found in a variety of source materials are merely illustrative of the application of the principles of the invention. It will be evident from the foregoing description that changes in the form, methods of use, and applications of the elements of the disclosed method for the improved methods of whole and half-antibody separation and purification use of are novel and may be modified and/or resorted to without departing from the spirit of the invention, or the scope of the appended claims.

Literature Cited and Incorporated by Reference:

1. MOLECULAR CLONING A LABORATORY MANUAL, 2nd Ed., ed. by Sambrook, Fritsch and Maniatis (Cold Spring Harbor Laboratory Press: 1989.

2. NA CLONING, Volumes I and II (D. N. Glover ed., 1985).

3. NUCLEIC ACID HYBRIDIZATION (B. D. Hames & S. J. Higgins eds. 1984).

4. TRANSCRIPTION OND TRANSLATION (B. D. Hames & S. J. Higgins eds. 1984).

5. CULTURE OF ANIMAL CELLS (R. I. Freshney, Alan R. Liss, Inc., 1987).

6. King DJ, et al., Expression, Purification And Characterization Of A Mouse-Human Chimeric Antibody And Chimeric Fab′ Fragment, BIOCHEM J. 1992 Jan 15; 281(Pt 2):317-23.

7. IMMOBILIZED CELLS AND ENZYMES (IRL Press, 1986).

8. B. Perbal, A PRACTICAL GUIDE To MOLECULAR CLONING (1984).

9. METHODS IN ENZYMOLOGY the treatise (Academic Press, Inc., N.Y.).

10. GENE TRANSFER VECTORS FOR MAMMALIAN CELLS (J. H. Miller and M. P. Calos eds., 1987, Cold Spring Harbor Laboratory).

11. METHODS IN ENZYMOLOGY, Vols. 154 and 155 (Wu et al. eds.).

12. Immunochemical Methods In Cell And Molecular Biology (Mayer and Walker, eds., Academic Press, London, 1987).

13. HANDBOOK OF EXPERIMENTAL IMMUNOLOGY, Volumes I-IV (D. M. Weir and C. C. Blackwell, eds., 1986).

14. MANIPULATING THE MOUSE EMBRYO, (Cold Spring Harbor Laboratory Press, Cold Spring Harbor, N.Y., 1986).

15. U.S. Pat. No., 6,441,145, Transgenically produced Antithrombin III

16. U.S. Pat. No., 6,268,487 Purification of biologically active peptides from milk

17. U.S. Pat. No., 5,849,992 Transgenic production of antibodies in milk

18. U.S. Pat. No., 5,849,992 Transgenic production of antibodies in milk

What is claimed is:

1. A method for separating IgG half antibodies from IgG whole antibodies, wherein the half antibodies and the whole antibodies are of the same isotype, comprising:
obtaining a sample that contains a mixture of IgG half antibodies and IgG whole antibodies of the same isotype;
reducing the pH of the sample such that the half antibodies dissociate from one another to form a resulting solution; and
applying the resulting solution to a column that differentially retards the mobility of the IgG half antibodies and IgG whole antibodies.

2. The method of claim 1, wherein the column retains both the IgG half antibodies and the IgG whole antibodies present in the resulting solution.

3. The method of claim 2, wherein the column is an ion exchange column.

4. The method of claim 3, wherein the ion exchange column is a cation exchange column.

5. The method of claim 2 further comprising subjecting the column to conditions which selectively elute IgG half antibodies retained by the column.

6. The method of claim 5, wherein the conditions which selectively elute IgG half antibodies retained by the column comprise adding a buffer to the column such that the pH of the buffer present within the column is increased to a level sufficient to selectively elute the IgG half antibodies.

7. The method of claim 6, wherein the pH of the buffer present within the column is increased to about 7.0 or greater.

8. The method of claim 5 further comprising subjecting the column to conditions which elute IgG whole antibodies retained by the column.

9. The method of claim 8, wherein the conditions which elute IgG whole antibodies comprise adding a buffer to the column such that the ionic strength of the buffer present within the column is increased to a level sufficient to elute the IgG whole antibodies.

10. The method of claim 1, wherein the IgG half antibodies and the IgG whole antibodies are of the IgG4 isotype.

11. The method of claim 1, wherein the IgG half antibodies and the IgG whole antibodies are of the IgG1, IgG2, or IgG3 isotype.

12. The method of claim 1, wherein the IgG half antibodies and the IgG whole antibodies are mammalian IgG half antibodies and IgG whole antibodies.

13. The method of claim 12, wherein the mammalian IgG half antibodies and IgG whole antibodies are human IgG half antibodies and IgG whole antibodies.

14. The method of claim 12, wherein the mammalian IgG half antibodies and IgG whole antibodies are chimeric IgG half antibodies and IgG whole antibodies.

15. The method of claim 12, wherein the mammalian IgG half antibodies and IgG whole antibodies are F(ab)₂ half antibodies and F(ab)₂ whole antibodies.

16. The method of claim 1, wherein the sample is obtained from milk.

17. The method of claim 16, wherein the milk is from a mammal.

18. The method of claim 16, wherein the milk is from an ungulate, pig, rabbit, or mouse.

19. The method of claim 1, wherein the sample is obtained from an egg.

20. The method of claim 1, wherein the sample is obtained from serum.

21. The method of claim 1, wherein the sample is obtained from cell culture medium.

22. A purified IgG half antibody preparation obtained by the method of claim 1.

23. The purified IgG half antibody preparation of claim 22, wherein the antibodies are of the IgG4 isotype.

24. The purified IgG half antibody preparation of claim 22, wherein half antibodies comprise at least 90% of the total amount of antibody in the preparation.

25. The purified IgG half antibody preparation of claim 24, wherein half antibodies comprise at least 95% of the total amount of antibody in the preparation.

26. The purified IgG half antibody preparation of claim 25, wherein half antibodies comprise at least 99% of the total amount of antibody in the preparation.

27. A purified IgG whole antibody preparation obtained by the method of claim 1, wherein the whole antibodies comprise a greater portion of the total antibody in the preparation as compared to the sample prior to being treated by the method of claim 1.

28. The purified IgG whole antibody preparation of claim 27, wherein the antibodies are of the IgG4 isotype.

29. The purified IgG whole antibody preparation of claim 27, wherein whole antibodies comprise at least 80% of the total antibodies in the preparation.

30. The purified IgG whole antibody preparation of claim 29, wherein whole antibodies comprise at least 90% of the total antibodies in the preparation.

31. A method for separating IgG half antibodies from IgG whole antibodies, wherein the half antibodies and the whole antibodies are of the same isotype, comprising:
obtaining a sample that contains a mixture of IgG half antibodies and IgG whole antibodies of the same isotype;
reducing the pH of the sample such that the half antibodies dissociate from one another to form a resulting solution;
applying the resulting solution to an ion exchange column such that both the IgG half antibodies and IgG whole antibodies are retained by the column;
adding a buffer to the column such that the pH of the buffer present within the column increases to a level sufficient to selectively elute the IgG half antibodies; and
subsequently adding a buffer to the column such that the ionic strength of the buffer present within the column increases to an amount sufficient to elute the IgG whole antibodies.

32. The method of claim 31, wherein the sample is obtained from milk.

33. The method of claim 32, wherein the milk is from a mammal.

34. The method of claim 33, wherein the milk is from an ungulate, pig, rabbit, or mouse.

35. The method of claim 31, wherein the sample is obtained from an egg.

36. The method of claim 31, wherein the sample is obtained from serum.

37. The method of claim 31, wherein the sample is obtained from cell culture medium.

38. The method of claim 31, wherein the IgG half antibodies and the IgG whole antibodies are of the IgG4 isotype.

39. The method of claim 31, wherein the pH of the sample is reduced to a pH below 4.0.

40. The method of claim 36, wherein the pH is reduced to a pH between about 2.0 to 4.0.

41. The method of claim 40, wherein the pH is reduced to a pH of about 3.5.

42. The method of claim 31, wherein the ion exchange column is a cation exchange column.

43. The method of claim 31, wherein the pH of the buffer present within the column is increased to at least 6.5 or greater.

44. The method of claim 43, wherein the pH of the buffer present within the column is increased to about 7.0.

45. A purified IgG half antibody preparation obtained by the method of claim 31.

46. The purified half antibody preparation of claim 45, wherein the antibodies are of the IgG4 isotype.

47. The purified half antibody preparation of claim 45, wherein half antibodies comprise at least 90% of the total amount of antibody in the preparation.

48. The purified half antibody preparation of claim 47, wherein half antibodies comprise at least 95% of the total amount of antibody in the preparation.

49. The purified half antibody preparation of claim 48, wherein half antibodies comprise at least 99% of the total amount of antibody in the preparation.

50. A purified IgG whole antibody preparation obtained by the method of claim 31, wherein the whole antibodies comprise a greater potion of the total antibody in the preparation as compared to the sample prior to being treated by the method of claim 31.

51. The purified IgG whole antibody preparation of claim 50, wherein the antibodies are of the IgG4 isotype.

52. The purified IgG whole antibody preparation of claim 50, wherein the whole antibodies comprise at least 80% of the total antibodies in the preparation.

53. The purified IgG whole antibody preparation of claim 52, wherein the whole antibodies comprise at least 90% of the total antibodies in the preparation.

54. A purified IgG half antibody preparation, wherein at least 90% of the total antibodies in the preparation are half antibodies.

55. A purified IgG whole antibody preparation, wherein the preparation includes half antibodies and whole antibodies and wherein at least 80% of the total antibodies are whole antibodies.

56. The preparation of claim 55, wherein the preparation further contains casein contaminants.

57. The method of claim 1, wherein said column is a HIC column.

58. The method of claim 31, wherein said column is a HIC column.

59. The method of claim 2, wherein said column is a HIC column.

60. The method of claim 5, wherein said column is a HIC column.

61. The method of claim 6, wherein said column is a HIC column.

62. The method of claim 43, wherein said column is a HIC column.
